(19) United States
(12) Patent Application Publication
(10) Pub. No.: US 2011/0004153 A1
(43) Pub. Date: Jan. 6, 2011

(54) CATHETER TIME MONITORING SYSTEMS AND METHODS OF USING THE SAME

(76) Inventor: ANITA KIPPING, Cumming, GA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(21) Appl. No.: 12/830,859

(22) Filed: Jul. 6, 2010

Related U.S. Application Data
(60) Provisional application No. 61/223,327, filed on Jul. 6, 2009.

(51) Int. Cl. A61M 25/00

(52) U.S. Cl. 604/65

(57) ABSTRACT

The various embodiments of the present disclosure relate generally to catheter time monitoring systems and methods of using the same. More specifically, the systems and methods of the present disclosure are designed for monitoring the amount of time a catheter time monitoring system has been in use. In one embodiment, a catheter time monitoring system comprises: a catheter subsystem and a timing subsystem, wherein the timing subsystem is integrated with the catheter subsystem, and wherein the catheter time monitoring system allows for monitoring a length of time that the catheter time monitoring system has been in use.

### CROSS REFERENCE TO RELATED APPLICATION

This application claims, under 35 U.S.C. §119(e), the benefit of U.S. Provisional Application Ser. No. 61/223,327, filed 6 Jul. 2009, the entire contents and substance of which are hereby incorporated by reference as if fully set forth below.

### TECHNICAL FIELD OF THE INVENTION

The various embodiments of the present disclosure relate generally to catheter time monitoring systems and methods of using the same. More specifically, the systems and methods of the present disclosure are designed for monitoring the amount of time a catheter time monitoring system has been in use.

### BACKGROUND OF THE INVENTION

The process used by hospitals to manage the duration of time a catheter system has been in use on a patient is relevant not only to the health and well-being of patients but also to the hospital's liability. Catheter systems are a fertile source of hospital-acquired patient infections. The longer catheter systems are in use, the greater the risk of culturing infectious bacteria. Thus, the risk of hospital-acquired patient infection is directly related to the ability of hospital personnel to accurately measure the time period from initialization of a patient's catheter system to the expiration of the period of use for the catheter system. Failure to replace an expired catheter system increases the risk of infection to the patient as well as the hospital's acquired liability. In fact, some insurance companies are no longer willing to pay for treatment of hospital-acquired infections, which requires hospitals to incur these expenses. Therefore, systems and methods of accurately and reliably monitoring the amount of time a particular catheter system has been in use on a patient are needed.

Many problems currently exist for nurses and other medical personnel when attempting to accurately calculate how long the same catheter system has been used on any given patient. Currently, hospitals measure the usage time of catheter systems by manually applying labels to catheter systems when they are initialized. This process presents many problems, including: nurses failing to apply labels once a catheter system is initialized; labels falling off, or otherwise becoming separated from, the catheter system; and labels being inaccurate. This process is so inconsistent that hospitals use periodic self-audits to evaluate the efficiency and accuracy of this process. Therefore, the current process used by hospitals is inadequate to accurately and reliably measure the time a catheter system has been in use, placing hospitals at increased risk for liability and patients at increased risk of infection.

Accordingly, there is a need for automated systems and methods of accurately monitoring the length of time that a catheter system has been in use. It is to the provision of such systems and methods that the various embodiments of the present invention are directed.

### BRIEF SUMMARY OF THE INVENTION

The various embodiments of the present disclosure relate generally to catheter time monitoring systems and methods of using the same. More specifically, the systems and methods of the present disclosure are designed for monitoring the amount of time a catheter time monitoring system has been in use.

An aspect of the present invention is a catheter time monitoring system comprising a catheter subsystem and a timing subsystem, where the timing subsystem is integrated with the catheter subsystem, and where the catheter time monitoring system allows for monitoring a length of time that the catheter time monitoring system has been in use. In some embodiments of the present invention, the length of time that the catheter system has been in use ranges from about 0 minutes to about 1 week or more.

The catheter subsystem can be an intravenous catheter In some embodiments, the catheter time monitoring system can further comprise a flow-regulating element in communication with the timing subsystem, where the flow-regulating element can allow the flow of a fluid through the system at substantially the same time as an activation of the timing subsystem, and where the flow-regulating element can disrupt the flow of a fluid through the system upon a passage of an amount of time, where the amount of time is determined by the timing subsystem. In some embodiments, the amount of time determined by the timing subsystem is about 72 hours. The flow-regulating element can be a valve or membrane, among others. In some embodiments of the present invention, the catheter time monitoring system can further comprise a notification element, where the passage of an amount of time activates the notification element.

Another aspect of the present invention comprises a method for determining a length of time a catheter time monitoring system has been in use. The method can comprise providing a subject with a catheter time monitoring system, where the system comprises a catheter subsystem a timing subsystem, where the timing subsystem is integrated with the catheter subsystem, and where the catheter time monitoring system allows for monitoring a length of time that the catheter time monitoring system has been in use. The method can further comprise activating the timing subsystem and determining the length of time that the catheter time monitoring system has been in use, where the activating a timing subsystem occurs at substantially the same time as the providing a subject with a catheter time monitoring system. In some embodiments, the length of time that the catheter time monitoring system has been in use ranges from about 0 minutes to about 1 week.

In some embodiments of the methods of the present invention, the catheter time monitoring system can further comprise a flow regulating element, where the flow regulating element is in communication with the timing subsystem. In some embodiments, activating the timing subsystem can comprise allowing flow of a fluid through the catheter time monitoring system.

In one embodiment, the method can further comprise allowing a passage of an amount of time, where the amount of time is determined by the timing subsystem, and disrupting flow of a fluid through the catheter time monitoring system upon the passage of the amount of time. In some embodiments, the amount of time determined by the timing subsystem is about 72 hours.

In an exemplary embodiment of the present invention, a catheter time monitoring system can comprise a catheter subsystem and a timing subsystem, where the timing subsystem is integrated with the catheter subsystem, and where the catheter time monitoring system allows for monitoring a length of time that the catheter time monitoring system has been in use, and where the amount of time determined by the timing subsystem is about 72 hours. In another exemplary embodiment, the catheter time monitoring system can further comprise a flow-regulating element in communication with the timing subsystem, where the flow-regulating element allows flow of a fluid through the catheter subsystem at substantially the same time that the timing subsystem is activated. In yet another exemplary embodiment, the catheter time monitoring system can further comprise a notification element in communication with the timing subsystem, where the passage of about 72 hours activates the notification element. In still yet another exemplary embodiment, the timing subsystem can comprise a display that displays the length of time that the catheter time monitoring system has been in use.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1 is a perspective view of a catheter time monitoring system with a flow-regulating element in fluid communication with a fluid channel of a spike.

FIG. 2 is a perspective view of a catheter time monitoring system with a flow-regulating element in fluid communication with a spike and a fluid chamber.

FIG. 3 is a perspective view of a catheter time monitoring system with a flow-regulating element in fluid communication with a fluid chamber and a catheter tube.

FIG. 4 is a perspective view of a catheter time monitoring system with a flow-regulating element in mechanical communication with a timing subsystem.

FIG. 5 is a perspective view of a catheter time monitoring system with a flow-regulating element in electrical communication with a timing subsystem.

FIG. 6 is a perspective view of a catheter time monitoring system with a flow-regulating element in mechanical communication with a timing subsystem in mechanical communication with a button.

FIG. 7 is a perspective view of a catheter time monitoring system with a flow-regulating element in mechanical communication with a timing subsystem in mechanical communication with a mechanical lever.

FIG. 8 is a perspective view of a catheter time monitoring system with a flow-regulating element altered to allow flow of a fluid through the system when a spike is maneuvered in the direction of a second end of a fluid chamber.

### DETAILED DESCRIPTION OF THE INVENTION

A major problem for hospitals and other medical facilities with systems for administering a catheter to a patient is monitoring the amount of time that a particular system has been in use. Accordingly, one aspect of this invention is a catheter time system capable of measuring the amount of time that a system has been in use. Further, systems of the present invention comprise initializing a timing subsystem at substantially the same time as allowing the flow of a fluid through the catheter system, and such systems are capable of more accurately determining the amount of time a catheter system has been in use as compared to other known systems. Accordingly, some embodiments of the invention provide a catheter system, which initializes a timing subsystem at substantially the same time as allowing a fluid to flow through a flow-regulating element. Further, continued use of a catheter system after passage of the system's expiration period of use can result in infection. Accordingly, other aspects of the invention are catheter systems that disrupt the flow of a fluid through a system after the passage of an amount of time, where the amount of time is determined by the timing subsystem.

Referring now to the figures, wherein like reference numerals represent like parts throughout the several views, exemplary embodiments of the present invention will be described in detail. Throughout this description, various components may be identified having specific values or parameters; however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another. Further, the terms “a,” “an,” and “the” do not denote a limitation of quantity, but rather denote the present of “at least one” of the referenced item.

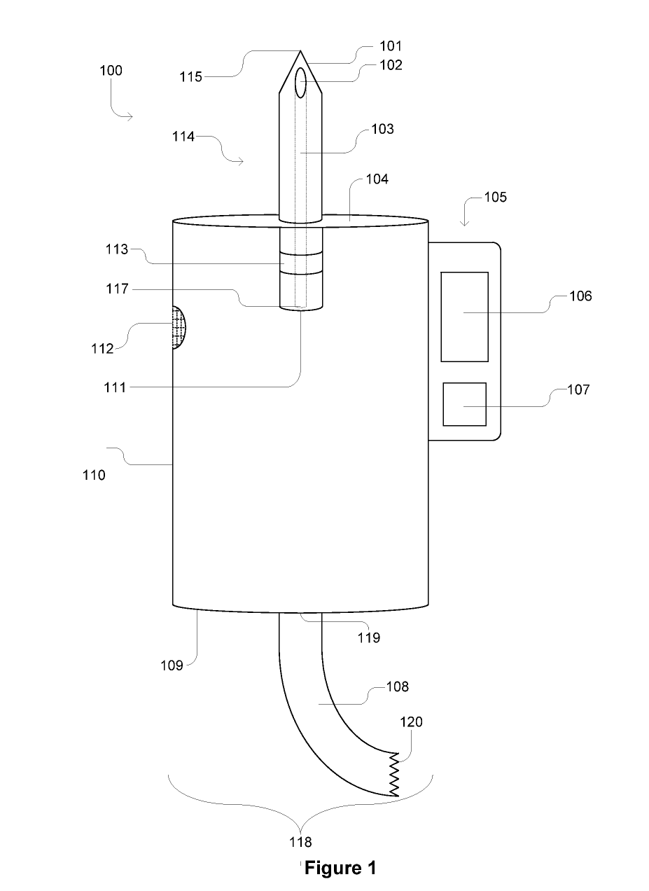

As shown in FIG. 1, one aspect of the present invention is a catheter time monitoring system 100. The system 100 can comprise a catheter subsystem 118 and a timing subsystem 105, where the timing subsystem 105 is integrated with the catheter subsystem 118, and where the catheter time monitoring system 100 allows for monitoring a length of time that the catheter time monitoring system 100 has been in use.

The catheter subsystem 118 can include many catheters known in the art. A catheter can be any tube that can be inserted into a body cavity, duct, vessel, among other of a subject, including, but not limited to, a urinary catheter, an intravenous drip catheter, a peripheral catheter, or the like. In an exemplary embodiment, the catheter subsystem 118 is an intravenous catheter. The catheter subsystem 118 can comprise a fluid chamber 110, a spike 114, a vent 112, and a catheter tube 108. The fluid chamber 110 can comprise a first end 104 and a second end 109. The spike 114 of the catheter subsystem 118 can comprise a first end 101, a second end 111, and a fluid channel 103. The first end 101 can comprise a puncture tip 115. The puncture tip 115 can comprise a fluid inlet 102. The second end 111 can comprise a fluid outlet 117. The fluid channel 103 provides fluid communication through the spike 114 from the first end 101 to the second end 111. The second end 111 of the spike 114 can be connected to the first end 104 of the fluid chamber 110 so that the spike 114 is in fluid communication with the fluid chamber 110. The catheter tube 108 of the catheter element 118 can be connected to the second end 109 of the fluid chamber 110. The vent 112 can comprise a porous membrane. The vent 112 can be located between the first end 104 and second end 109 of the fluid chamber 110, such that the vent 112 can be in communication with the fluid chamber 110 and the ambient surroundings of the fluid chamber 110. The catheter tube 108 can comprise any flexible tube having a first end 119 and a second end 120 that is capable of transporting a fluid. The first end 119 of the catheter tube 108 can be in fluid communication with the second end 109 of the fluid chamber 110.

According to the various embodiments of the present invention, the catheter time monitoring system 100 comprises a timing subsystem 105. The timing subsystem 105 can comprise a timer 106, a notification element 107, and a linking element. The timer 106 can be any timer known in the art, including, but not limited to, a mechanical timer, an electrical timer, or combinations thereof. In an exemplary embodiment, the timer 106 can be an electrical timer. In another exemplary embodiment, the timer 106 can comprise a fluid reservoir, such as one manufactured by Brenmoor, Ltd., which, upon rupturing, causes a fluid (e.g. ink or colorimetric indicator) to migrate through a migration medium as time passes.

In some embodiments, the timer 106 can comprise a display for displaying the amount of time the catheter time monitoring system 100 has been in use. The display can be any display known in the art, including, but not limited to, a digital display, an analog display, or combinations thereof. In an exemplary embodiment, the display can be a digital display.

The timing subsystem 105 can further comprise a notification element 107 capable of indicating whether the usage time of a catheter time monitoring system 100 has reached its expiration period. The notification element 107 can be many known notification elements in the art, including, but not limited to, an audible alarm element, a visual alarm element, or combinations thereof. For example, where the notification element 107 comprises an audible alarm element, the audible alarm element can be many sound producing devices known in the art, such as a speaker. Where the notification element 107 comprises a visual alarm element, the visual alarm element can be many light producing devices known in the art, such as a light emitting diode.

The timing subsystem 105 can further comprise a linking element capable of allowing communication between the timer 106, the notification element 107, a remote processing unit, and various combinations thereof. The linking element can include, but is not limited to, a central processing unit, such as an embedded microcontroller. In some embodiments where the linking element comprises a central processing unit, the central processing unit can be capable of generating an electrical output signal, wherein the electrical output signal can be an analog electrical signal, a digital electrical signal, or a combination thereof. For example, the central processing unit can 1) communicate with the timer 106 to determine the amount of time the catheter time monitoring system 100 has been in use; 2) communicate with the notification element 107 to produce an audible alarm, visual alarm, or a combination thereof; and/or 3) communicate with a control system, such as a computer at a remote nursing station or an intravenous pump monitoring system, via electrical wiring or electromagnetic waves, or the like.

There are many ways in which the catheter subsystem 118 and the timing subsystem 105 can be integrated, including, but not limited to, mechanical integration, electrical integration, or combinations thereof. In some embodiments, the integration of the timing subsystem 105 and the catheter subsystem 118 occurs through mechanical integration, such that the catheter subsystem 118 and the timing subsystem 105 are in mechanical communication. For example, mechanical integration of the timing subsystem 105 with the catheter subsystem 118 can occur by physically coupling the two subsystems together, which may include, but is not limited to, any fasteners known in the art, such as a screw, a clamp, or a bolt, or any bonding adhesive known in the art, such as glue. In some embodiments, the integration of the timing subsystem 105 with the catheter subsystem 118 can occur through electrical integration, such that the timing subsystem 105 and the catheter subsystem 118 are in electrical communication. For example, electrical integration of the timing subsystem 105 with the catheter subsystem 118 can occur when the timing subsystem 105 and the catheter subsystem 118 are in electrical communication via electrical wiring. Further, electrical integration of the timing subsystem 105 with the catheter subsystem 118 can occur by when the timing subsystem 105 and the catheter subsystem 118 are in electrical communication via electromagnetic signals, such as radio frequency electromagnetic waves, transmitted between the timing subsystem 105 and the catheter subsystem 118. When electrical communication occurs through electrical wiring or electromagnetic signals, the timing subsystem 105 can be located remotely from the catheter subsystem 118, such as at a nursing station, an intravenous pump monitoring system, or the like.

The system 100 allows for the monitoring of a length of time that the system 100 has been in use. The length of time that the system 100 has been in use may range from about zero minutes to any selected time limit. In an exemplary embodiment, the length of time can range from zero minutes to about one week. In another exemplary embodiment, the length of time can range from about zero minutes to about seventy-two hours. In some embodiments, the length of time can be stored in a memory device by the timing subsystem 105. The timing subsystem 105 can comprise a display 106, which can show the length of time, flow rate, or fluid characteristics, among others. The length of time that can be displayed on the display 106 includes, but is not limited to, the length of time the system 100 has been in use, the length of time remaining until the system's period of use expires, or any combination thereof.

The catheter time monitoring system 100 can further comprise a flow-regulating element 113. The flow-regulating element 113 can be used to allow the flow of a fluid through the system 100, disrupt the flow of a fluid through the system 100, or a combination thereof. In some embodiments, the flow-regulating element 113 allows the flow of a fluid through the system 100 at substantially the same time that the timing subsystem 105 is activated. In some embodiments, the flow-regulating element 113 disrupts the flow of a fluid through the system 100 after the passage of an amount of time that is determined by the timing subsystem 105. The flow-regulating element 113 can be many flow-regulating elements known in the art, including, but not limited to, a valve (e.g. solenoid valve, globe valve, gate valve, among others), a membrane, or combinations thereof. For example, in an exemplary embodiment, the flow-regulating element 113 can comprise a valve that allows the flow of a fluid through the system 100 at substantially the same time as activation of the timing subsystem 105. In another exemplary embodiment, the flow-regulating element 113 can comprise a valve that disrupts the flow of a fluid through the system 100 after the passage of the expiration period of use for the system 100. In yet another exemplary embodiment, the flow-regulating element 113 can comprise a valve that allows the flow of a fluid through the system 100 at substantially the same time as activation of the timing subsystem 105 and disrupts the flow of a fluid through the system 100 after the passage of the expiration period of use for the system.

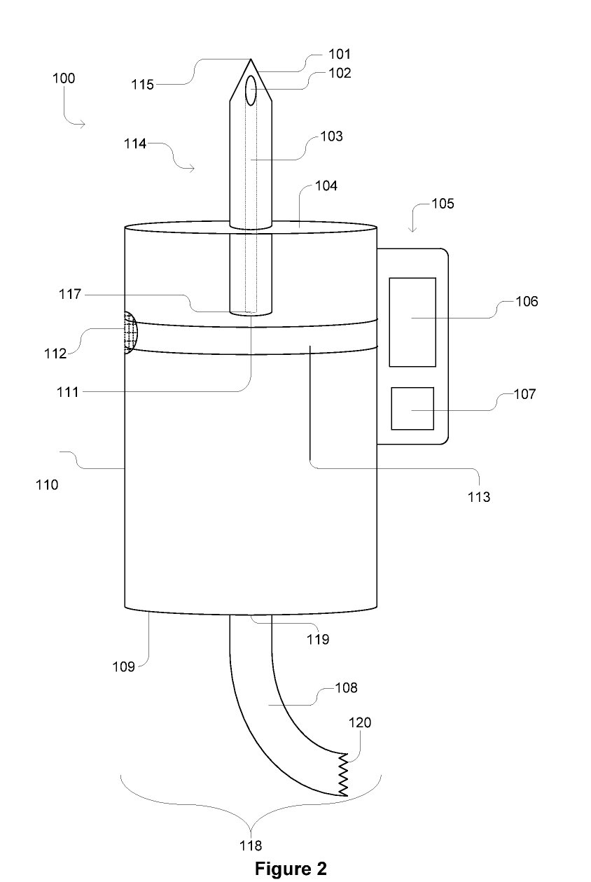

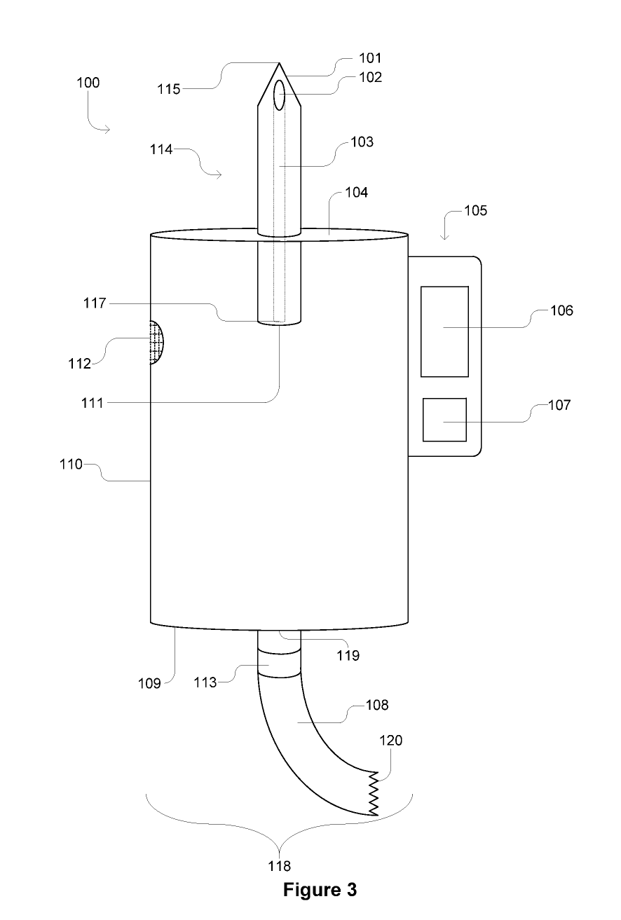

As illustrated in FIG. 1, in some embodiments, the flow-regulating element 113 can be in fluid communication with the fluid channel 103. When the flow-regulating element 113 is in fluid communication with the fluid channel 103, the flow-regulating element 113 can allow and/or disrupt the flow of fluid through the spike 114. As illustrated in FIG. 2, in some embodiments, the flow-regulating element 113 can be in fluid communication with the fluid chamber 110. When the flow-regulating element 113 is in fluid communication with the fluid chamber 110, the flow-regulating element 113 can allow and/or disrupt the flow of a fluid from the spike 114 to the fluid chamber 110. As illustrated in FIG. 3, in some embodiments, the flow-regulating element 113 can be in fluid communication with the catheter tube 108. When the flow-regulating element 113 is in fluid communication with the catheter tube 108, the flow-regulating element 113 can allow and/or disrupt the flow of a fluid from the fluid chamber 110 to the catheter tube 108. In an exemplary embodiment, the flow-regulating element 113 is in fluid communication with the fluid chamber 110 and allows and disrupts the flow of a fluid through the fluid chamber 110.

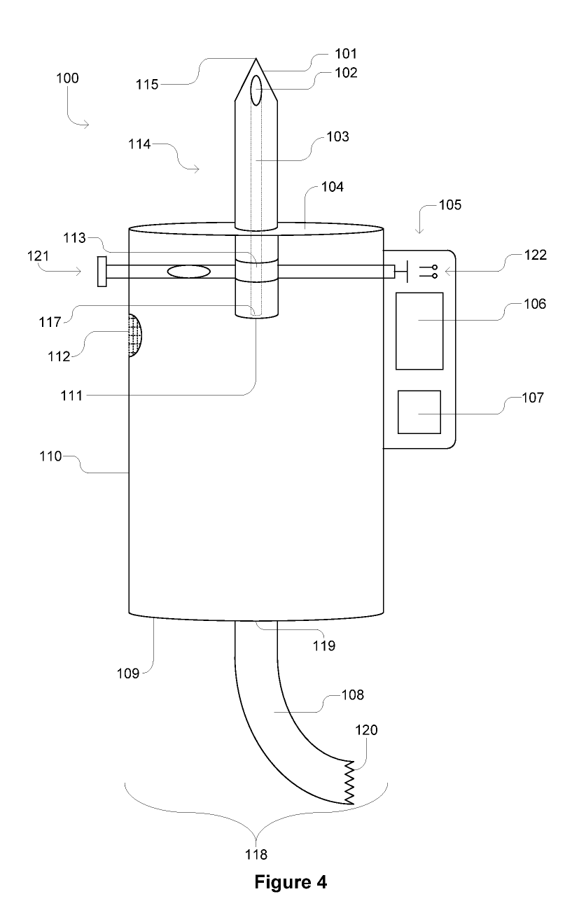

The flow-regulating element 113 can be in communication with the timing subsystem 105 in many ways, including, but not limited to, mechanical communication, electrical communication, or a combination thereof. When the flow-regulating element 113 is in mechanical communication with the timing subsystem 105, a mechanical element can activate the timing subsystem 105 at substantially the same time as a fluid begins to flow through the system 100. In an exemplary embodiment, as illustrated in FIG. 4, a mechanical member 121 is configured in a manner causing the flow regulating element 113 to prevent the flow of a fluid through the fluid channel 103, and the timing subsystem 105 is not activated. If the mechanical member 121 is maneuvered in a direction transverse to the fluid chamber 110 and the spike 114 towards the timing subsystem 105, the flow-regulating element 113 can allow flow of a fluid through the spike 114 at substantially the same time as the mechanical member 121 can close the circuit 122 in the timing subsystem 105, which would activate the timing subsystem 105.

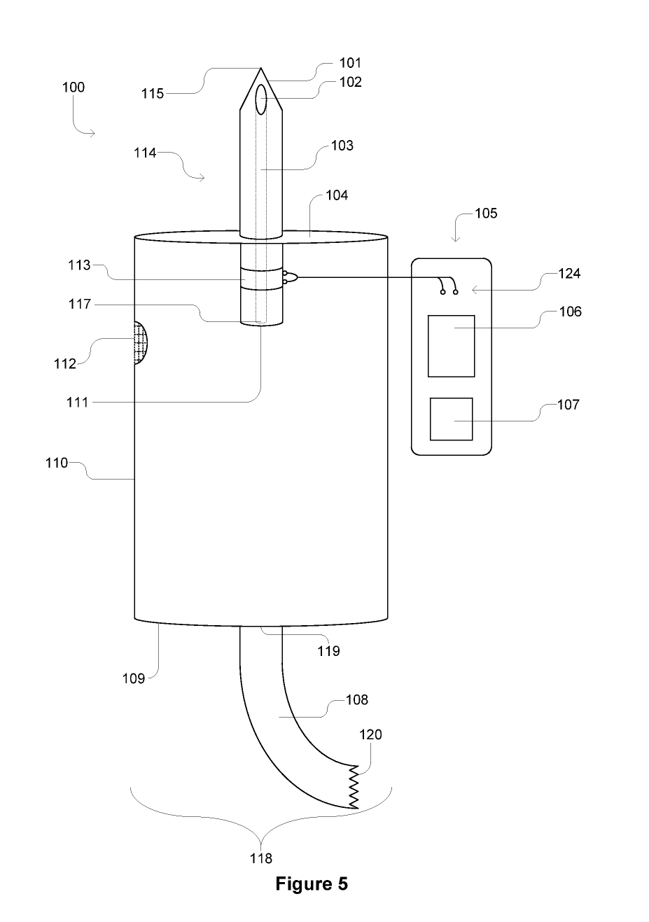

In some embodiments, the flow-regulating element 113 can be in electrical communication with the timing subsystem 105. When the flow-regulating element 113 is in electrical communication with the timing subsystem 105, an electrical signal can be sent from the timing subsystem 105 to the flow-regulating element 113 that allows or disrupts the flow of a fluid through the system 100. The electrical signal can be sent from the timing subsystem 105 to the flow-regulating element 113 by many ways known in the art, including, but not limited to, electrical wiring, electromagnetic signals, such as radio frequency electromagnetic waves, or any combination thereof. For example, as illustrated in FIG. 5, the timing subsystem 105 is in electrical communication with the flow-regulating element 113 via electrical wiring 124. In one example, the timing subsystem 113 can be disconnected and reconnected to the flow-regulating element 113. In another example, the timing subsystem 105 is continuously integrated with the flow-regulating element 113. An electrical signal can be sent from the timing subsystem 105 to the flow-regulating element 113 that will alter the flow-regulating element 113 to either allow the flow of a fluid through the system 100 at substantially the same time as activation of the timing subsystem 105 or disrupt the flow of a fluid through the system 100 after the passage of an amount of time determined by the timing subsystem 105, such as the period of use for a particular catheter time monitoring system 100.

In some embodiments of the present invention, the catheter time monitoring system 100 further comprises a flow regulating element 113, where the flow-regulating element 113 allows the flow of a fluid through the system 100 to occur at substantially the same time as the activation of the timing subsystem 105. Activation of the timing subsystem 105 can occur in many ways, including, but not limited to, pressing a button, manipulating a lever, inserting a spike 114 into a catheter fluid reservoir, such as an intravenous drip bag, transmitting an electrical signal to the timing subsystem 113, or any combination thereof.

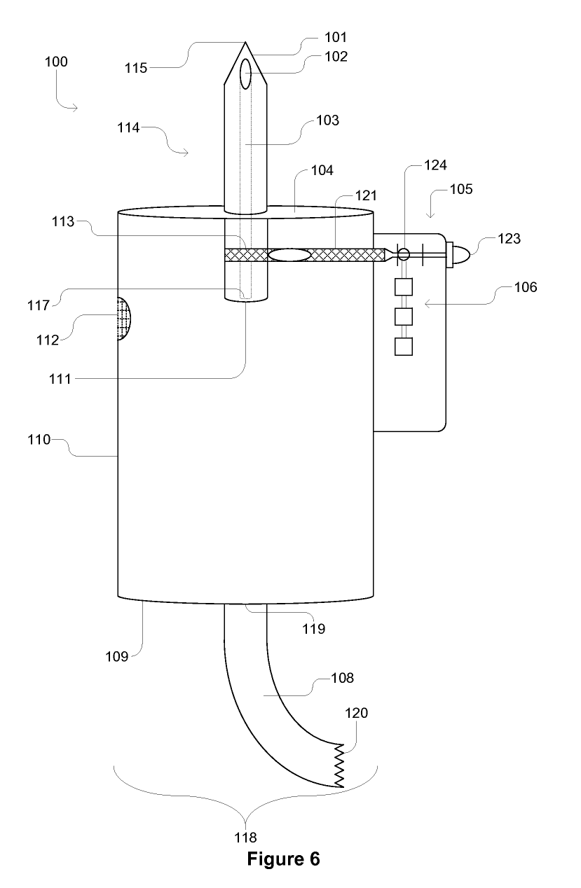

In an exemplary embodiment, as illustrated in FIG. 6, the flow-regulating element 113 is altered to allow the flow of a fluid through the system 100, and the timing subsystem 105 is activated at substantially the same time by pressing a button 123 on the timing subsystem 105. As shown in FIG. 6, by pressing a button 123 on the timing subsystem 105, a mechanical member 121 is maneuvered transverse to the fluid chamber 110 and the spike 114 in the direction of the spike 114 to alter the flow-regulating element 113, which allows the flow of a fluid through the system 100. At substantially the same time as the flow-regulating element 113 allows flow of a fluid through the system 100, a reservoir of fluid 124 (e.g. ink or colorimetric indicator) is ruptured, which activates the timing subsystem 105. The ink then migrates through a migration medium in the direction of the display 106. The distance that the ink migrates corresponds to the amount of time the system 100 has been in use.

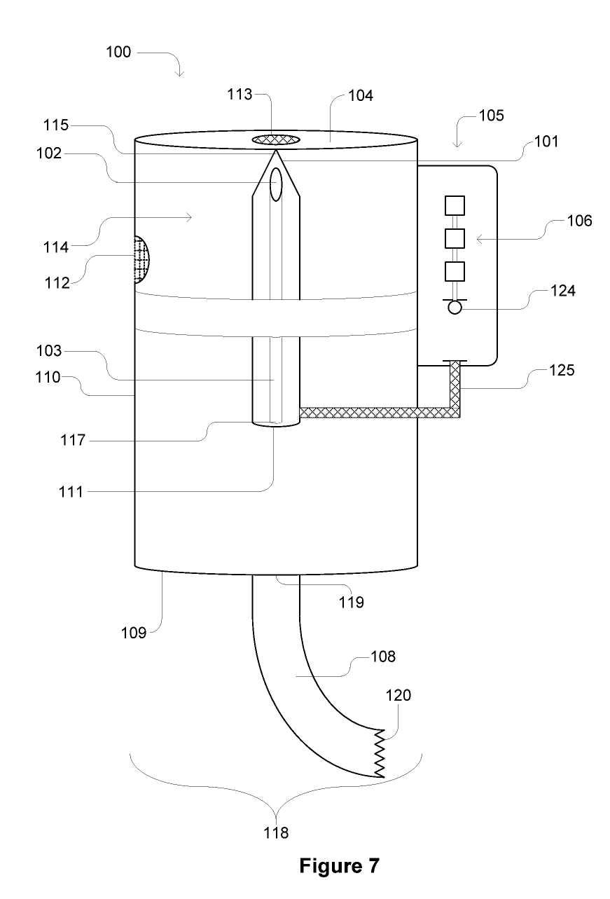

In another exemplary embodiment, as illustrated in FIG. 7, the flow-regulating element 113 is altered to allow the flow of a fluid through the system 100, and the timing subsystem 105 is activated at substantially the same time by maneuvering a mechanical lever 125 in the direction of the first end 104 of the fluid chamber 110. Before maneuvering the mechanical lever 125, the spike 114 is located substantially within the fluid chamber 110. When the mechanical lever is maneuvered in the direction of the first end 104 of the fluid chamber 110, the puncture tip 115 of the spike 114 alters the flow-regulating element 113, such that the fluid channel 103 of the spike 114 can be in fluid communication with a catheter fluid reservoir (e.g., an intravenous drip bag), thus allowing the flow of a fluid through the system 100 at substantially the same time as the reservoir of fluid 124 is ruptured, which activates the timing subsystem 105.

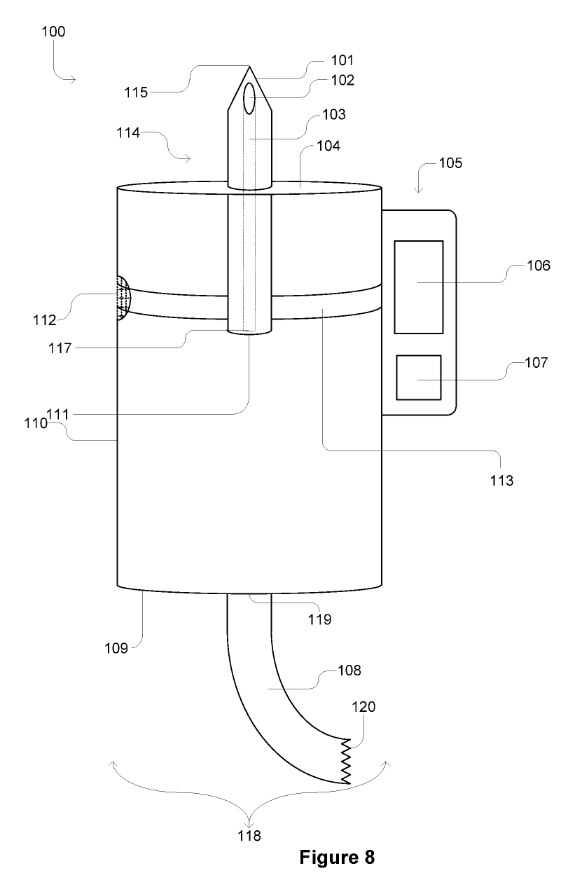

In yet another exemplary embodiment, the system 100 is initially set up, as illustrated in FIG. 2, so that the flow-regulating element 113 disrupts the flow of a fluid through the system 100. In such an embodiment, as further illustrated in FIG. 8, when the spike 114 is maneuvered in the direction of the second end 109 of the fluid chamber 110, the second end 111 of the spike 114 alters the flow-regulating element 113 to allow the flow of a fluid through the system 100. At substantially the same time as the flow-regulating element 113 is altered to allow the flow of a fluid through the system 100, the reservoir of fluid 124 is ruptured, which activates the timing subsystem 105. In such embodiments, the spike 114 can be retractable, adjustable, spring-loaded, or the like.

In still yet another exemplary embodiment, the flow-regulating element 113 is altered to allow the flow of a fluid through the catheter time monitoring system 100 by maneuvering a lever or twisting an activation device on the catheter subsystem 118. In this exemplary embodiment, the catheter subsystem 118 can comprise a peripheral catheter. Further, the maneuvering a lever or twisting an activation device can also expose a needle, which can be inserted into a patient, at substantially the same time as the timing subsystem 105 is activated.

The catheter time monitoring system 100 that further comprise a flow-regulating element 113 can allow the flow-regulating element 113 to disrupt the flow of a fluid through the system 100 upon the passage of an amount of time, where the amount of time is determined by the timing subsystem 105. The amount of time allowed to pass before flow of a fluid is disrupted can be any selected amount of time, including, but not limited to, the amount of time a system 100 may be in use before reaching its designated expiration time. In an exemplary embodiment, the flow-regulating element 113 disrupts the flow of a fluid through the system 100 upon the passage of an amount of time equal to about seventy-two hours. The amount of time that can pass before disrupting the flow of a fluid through the system can be determined by the timing subsystem 105 in many ways, including, but not limited to, medical personnel inputting a desired amount of time into the timing subsystem 105 prior to using the system 100, manufacturing the system 100 with a predetermined amount of time loaded into the timing subsystem 105, or combinations thereof. In an exemplary embodiment, the system 100 is manufactured with a predetermined amount of time allowed to pass before the flow-regulating element 113 disrupts the flow of fluid through the system 100.

Another aspect of this invention is a method of monitoring the length of time that the catheter time monitoring system 100 has been in use. The method can comprise providing a subject with a catheter time monitoring system 100, activating a timing subsystem 105, and determining the length of time the system 100 has been in use.

A used herein, the term “subject” refers to a vertebrate, preferably a mammal, and more preferably a human. Mammals include, but are not limited to, primates, humans, cows, dogs, mice, rabbits, swine, rats, guinea pigs and equine.

Activating a timing subsystem 105 can be performed manually, automatically, or a combination thereof. When a timing subsystem 105 is activated manually, the activation may occur many ways, including, but not limited to pressing a button, manipulating a lever, or combinations thereof. When a timing subsystem 105 is activated automatically, the timing subsystem 105 is activated at substantially the same time the flow of a fluid through the system 100 begins. A timing subsystem 105 may be automatically activated many ways, including, but not limited to altering the flow regulating element 113 to allow flow of a fluid through the system 100, inserting the spike 114 into a catheter fluid reservoir (e.g., an intravenous drip bag), or combinations thereof. Determining the length of time that the system 100 has been in use can be done many ways, including, but not limited to, monitoring the display 106, monitoring the notification element 107, or combinations thereof. In an exemplary embodiment, a person determines the length of time that the system has been in use by monitoring the display 106 and the notification element 107.

Some embodiments of the present invention that are directed to a method of monitoring the amount of time a catheter time monitoring system 100 has been in use can further comprise allowing the passage of an amount of time and disrupting the flow of a fluid through the catheter time monitoring system 100 upon the passage of the amount of time. In some of these embodiments, the amount of time can be determined by the timing subsystem 105. The flow of a fluid through the system 100 can be disrupted many ways known in the art, including, but not limited to, manually removing the system 100 from use, manually altering the flow-regulating element 113, automatically altering the flow-regulating element 113 by communication with the timing subsystem 105, or combinations thereof. Manually altering the flow-regulating element 113 can include any process that involves any substantial human intervention to stop the flow of a fluid through the system 100. Automatically altering the flow-regulating element 113 can include any process that can be performed without substantial human intervention after the passing of the amount of time, including, but not limited to, transmitting an electric or electromagnetic signal from the timing subsystem 105 to the flow-regulating element 113. In an exemplary embodiment, the flow of a fluid through the catheter time monitoring system 100 can be automatically disrupted by transmitting an electrical signal from the timing subsystem 105 to the flow-regulating element 113 after the passage of an amount of time equal to about seventy-two hours.

It must be noted that, as used in this specification and the appended claims, the singular forms “a,” “an,” and “the” include plural referents unless the context clearly dictates otherwise.

All patents, patent applications, and references included herein are specifically incorporated by reference in their entireties.

The present invention is illustrated by way of the examples contained herein, which are provided for clarity of understanding. The exemplary embodiments should not be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

What is claimed is:

1. A catheter time monitoring system, comprising:
a catheter subsystem; and,
a timing subsystem, wherein the timing subsystem is integrated with the catheter subsystem, and
wherein the catheter time monitoring system allows for monitoring a length of time that the catheter time monitoring system has been in use.

2. The catheter time monitoring system of claim 1, further comprising a flow-regulating element in communication with the timing subsystem.

3. The catheter time monitoring system of claim 2, wherein the flow-regulating element allows flow of a fluid through the system at substantially the same time as an activation of the timing subsystem

4. The catheter time monitoring system of claim 3, further comprising a notification element, wherein the passage of an amount of time activates the notification element.

5. The catheter time monitoring system of claim 2, wherein the flow-regulating element disrupts flow of a fluid through the system upon a passage of an amount of time, and wherein the amount of time is determined by the timing subsystem.

6. The catheter time monitoring system of claim 5, further comprising a notification element, wherein the passage of the amount of time activates the notification element.

7. The catheter time monitoring system of claim 1, wherein the catheter subsystem is an intravenous catheter.

8. The catheter time monitoring system of claim 2, wherein the flow-regulating element is a valve or a membrane.

9. The catheter time monitoring system of claim 1, wherein the length of time that the catheter time monitoring system has been in use ranges from about 0 minutes to about 1 week.

10. The catheter time monitoring system of claim 1, wherein the amount of time determined by the timing subsystem is about 72 hours.

11. A method for determining a length of time a catheter time monitoring system has been in use, the method comprising:
providing a subject with a catheter time monitoring system comprising a catheter subsystem a timing subsystem, wherein the timing subsystem is integrated with the catheter subsystem, and wherein the catheter time monitoring system allows for monitoring a length of time that the catheter time monitoring system has been in use;
activating the timing subsystem; and
determining the length of time that the catheter time monitoring system has been in use,
wherein the activating a timing subsystem occurs at substantially the same time as the providing a subject with a catheter time monitoring system.

12. The method of claim 11, wherein the activating the timing subsystem comprises allowing flow of a fluid through the catheter time monitoring system.

13. The method of claim 11, wherein the catheter time monitoring system further comprises a flow regulating element, wherein the flow regulating element is in communication with the timing subsystem.

14. The method of claim 13, wherein activating the timing subsystem comprises allowing the flow of a fluid through the catheter subsystem.

15. The method of claim 13, further comprising:
allowing a passage of an amount of time, wherein the amount of time is determined by the timing subsystem; and
disrupting flow of a fluid through the catheter time monitoring system upon the passage of the amount of time.

16. The method of claim 11, wherein the length of time that the catheter time monitoring system has been in use ranges from about 0 minutes to about 1 week.

17. The method of claim 15, wherein the amount of time determined by the timing subsystem is about 72 hours.

18. A catheter time monitoring system, comprising:
a catheter subsystem; and,
a timing subsystem, wherein the timing subsystem is integrated with the catheter subsystem, and wherein the catheter time monitoring system allows for monitoring a length of time that the catheter time monitoring system has been in use, and wherein the amount of time determined by the timing subsystem is about 72 hours,
a flow-regulating element in communication with the timing subsystem, wherein the flow-regulating element allows flow of a fluid through the catheter subsystem at substantially the same time that the timing subsystem is activated; and
a notification element in communication with the timing subsystem, wherein the passage of about 72 hours activates the notification element.

19. A catheter time monitoring system of claim 18, wherein the time system further comprises a display that displays the length of time that the catheter time monitoring system has been in use.

20. A catheter time monitoring system of claim 18, wherein the flow-regulating element disrupts flow of a fluid through the catheter subsystem upon a passage of an amount of time.
